(19) United States
(12) Patent Application Publication
(10) Pub. No.: US 2004/0266813 A1
(43) Pub. Date: Dec. 30, 2004

(54) Injectable sustained-release microspheres of huperzine a compoounds

(76) Inventors: Wanhui Liu, Yantai City (CN); Jilun Song, Yantai City (CN); Dafeng Chu, Yantai City (CN); Ke Liu, Yantai City (CN)

Correspondence Address: Thomas E Ciotti, Morrison & Foerster, 755 Page Mill Road, Palo Alto, CA, 94304, US

(21) Appl. No.: 10/482,631

(22) PCT Filed: Jul. 3, 2002
(86) PCT No.: PCT/CN02/00472

(30) Foreign Application Priority Data
Jul. 3, 2001 (CN) 0111995.0

(51) Int. Cl. A61K 9/26; A61K 31/473

(52) U.S. Cl. 514/295; 424/469

(57) ABSTRACT

Sustained-release microspheres of Huperzine A compounds, the preparation and use thereof, said sustained-release microspheres comprise Huperzine A compounds represented by the formula (Ia) and biodegradably pharmaceutically acceptable polymer excipients,

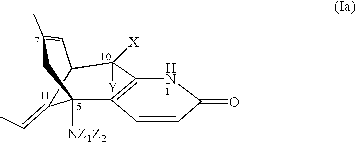

wherein, X and Y independently represent hydrogen or methyl, Z₁ and Z₂ independently represent hydrogen or in combination represent

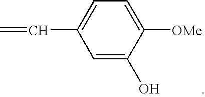

### FIELD OF THE INVENTION

The present invention relates to sustained-release microspheres for injection of Huperzine A or its derivatives or salts (hereinafter referred as “Huperzine A compounds”), more particularly, to sustained-release microspheres for injection of Huperzine A compounds and a method for preparing the same and a method for the treatment of disorders involving acetylcholinesterase by using said Huperzine A compounds formulation, and a method for the treatment of Alzheimer's disease.

### DESCRIPTION OF THE BACKGROUND ART

Huperzine A a formula (I), the IUPAC name thereof is (5R, 9R, 11E)-5-amino-11-ethylidene-5,6,9,10-tetrahydro-7-methyl-5,9-methlenecyclo octa[b]pyridin-2[1H]-one, an alkaloid obtainable from the plant Huperzia serrata (Thunb) Thev of Clubmoss Family (Lycopodiaceae Reichb.) (Zhongguo Yaoxue Zazhi, 32 (5), 260-261, 1997; WO92/19238, etc.).

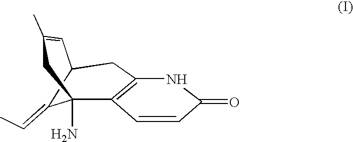

It was reported that Huperzine A is a highly effective and reversible acetylcholinesterase inhibitor (Zhongguo Yaoxue Zazhi, ibid; WO02/11712; EP806416A1, etc.), and is useful for the treatment of Alzheimer's disease (Zhongguo Yaoxue Zazhi, ibid; WO01/00215; WO02/11712, etc.). In addition, it was found via structural modification that some Huperzine A derivatives also have similar actions (WO99/11525; EP1167354A2; U.S. Pat. No. 5,869,672; U.S. Pat. No. 5,547,960; WO92/19238; EP806416A1, etc.).

It was known that the dosage forms of huperzine A are tablets, capsules, transdermal delivery systems (TDS) and injections, etc.(WO02/11712; CN1047732C; CN1279065A, etc.). However, various dosage forms of Huperzine A in clinical practice, no matter oral formulation, injection or TDS, have disadvantages in clinical practice. Since Huperzine A has a relative short of biological half life period and large side effect, such as dizzy, nausea, hidrosis, cathisophobia, dysphoria, vomit, muscular shaking, heart rate changing, and pupilla varying. At present, Huperzine A tablets are administrated 2-3 times per day and should be administrated over a long period, such cannot avoid the occurrence of side effects.

For patients suffering from Alzheimer's disease, it is apprently that it is unrealistic to take medicine on time and amount for a long time or inject everyday for a long time. Although TDS may realize long acting at certain extent (CN1047732C), its long acting effect is obviously insufficient to Alzheimer's disease, moreover, in order to achieve long effecting with TDS, it is necessary to increase dosage, this may cause local stimulation of skin. Therefore, there is urgent need of developing a novel long-effect formulation, which can reduce administration frequency and is convenient to patients, and has little probability to occur side effects.

### OBJECT OF THE INVENTION

The object of the present invention is to provide sustained-release microspheres for injection of Huperzine A compounds, which can reduce the frequency of administration from 2-3 times per day to once half month, one month, or even two months. As a result, times for administration are greatly reduced, and the life quality of patients suffering from Alzheimer's disease is greatly improved.

Another purpose of the present invention is to provide a method for preparation of said sustained-release microspheres for injection of Huperzine A compounds.

More another purpose of the present invention is to provide a method for the treatment of disorders involving acetylcholinesterase by using Huperzine A compounds formulation and a method for the treatment of Alzheimer's disease.

The above-mentioned purposes of the present invention can be achieved by the following technical solutions.

### SUMMARY OF THE INVENTION

The first aspect of the invention provides sustained-release microspheres of Huperzine A compounds, characterized in that the sustained-release microspheres comprise Huperzine A compounds represented by the formula (Ia) and biodegradably pharmaceutically acceptable polymer excipients,

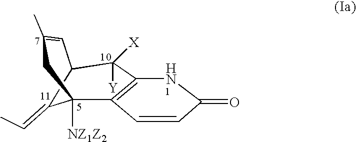

wherein, X and Y independently represent hydrogen or methyl, Z₁ and Z₂ independently represent hydrogen or in combination represent

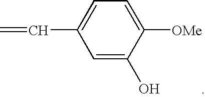

The second aspect of the invention provides a method for preparation of sustained-release microspheres of Huperzine A compounds, comprising dissolving Huperzine A compounds and biodegradably pharmaceutically acceptable excipients with an organic solvent, dropping organic solvent phase into an emulsion compounded with pharmaceutically acceptable water-soluble polymer to give sustained-release microspheres by solvent evaporation.

The third aspect of the invention provides another method for preparation of sustained-release microspheres of Huperzine A compounds, comprising dissolving Huperzine A compounds and biodegradably pharmaceutically acceptable polymer excipients with an organic solvent and spray drying to give the microspheres.

The fourth aspect of the invention provides a method for the treatment of Alzheimer's disease by using said sustained-release microspheres as described in the first aspect of the invention.

Other aspects and advantages of the invention will be apparent to those skilled in the art from the following detailed description of the invention.

### BRIEF DESCRIPTION OF THE DRAWINGS

FIG. 1. is the figure showing cumulative release rate of sustained-release microspheres obtained in Example 1 in a simulated-release solution of pH 4.

FIG. 2. is the figure showing release rate per day of sustained-release microspheres obtained in Example 1 in simulated-release solution of pH 4 during determination period.

FIG. 3. is the figure showing cumulative release rate of sustained-release microspheres obtained in Example 4 in simulated-release solution of pH 4.

FIG. 4. is the figure showing release rate per day of sustained-release microspheres obtained in Example 4 in simulated-release solution of pH 4 during determination period.

FIG. 5. is the figure showing cumulative release rate of sustained-release microspheres obtained in Example 4 in simulated-release solution of pH 5.5.

FIG. 6. is the figure showing release rate per day of sustained-release microspheres obtained in example 4 in simulated-release solution of pH 5.5 during determination period.

### DETAILED DESCRIPTION OF THE INVENTION

The sustained-release microspheres of Huperzine A compounds of the invention are consisted of Huperzine A compounds and biodegradably pharmaceutically acceptable polymer excipients.

The Huperzine A compounds of the invention are assemblies of compounds (Huperzine A, derivatives, analogues and pharmaceutically acceptable salts thereof) having structures of the following general formula (Ia):

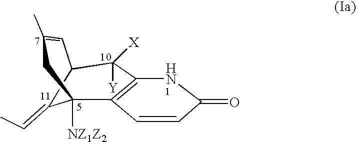

wherein, X and Y independently represent hydrogen or methyl, Z₁, and Z₂ independently represent hydrogen or together represent

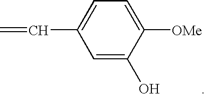

The sustained-release microspheres of the invention are suitable for all above-mentioned Huperzine A or derivatives or analogues and pharmaceutically acceptable salts thereof.

Preferably, said Huperzine A compounds are selected from at least one of the compounds (I), (II), (III), (IV) or (V) shown in the following table or their acid addition salts.

| Compounds | X | Y | Z₁Z₂ |
|---|---|---|---|
| I | H | H | H₂ |
| II | H | H | 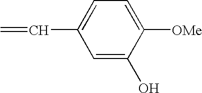 |
| III | H | CH₃ | H₂ |
| IV | CH₃ | H | H₂ |
| V | CH₃ | CH₃ | H₂ |

If the compound (I) is named from Huperzine A as parent core, the compound (II) is N5-(3′-hydroxy-4′-methoxy-phenylmethylene) Huperzine A, the compound III is (10S)-10-methyl Huperzine A, the compound (IV) is (10R)-10-methyl Huperzine A, and the compound (IV) is 10,10-dimethyl Huperzine A.

Among the above compounds, Huperzine A, i.e., the compound (I) is most preferred.

The above Huperzine A compounds may be used in the form of free base or acid addition salts. The acids to form the acid addition salts may be hydrochloric acid, acetic acid, phosphoric acid, sulfuric acid, lactic acid and citric acid, etc.

In the above sustained-release microspheres, the pharmaceutically acceptable polymer excipients may be biodegradable and water-insoluble polymeric materials, such as, including but not limited to poly(D, L-lactide-co-glycolide) (PLGA), poly(lactic acid), poly(glycollic acid), poly (3-hydroxybutyrate), polylactone, poly(acid anhydride), poly (hydroxybutyrate-hydroxyvalerate), poly(acrylic glucosan), poly(lactic acid)-polyethlyeneglycol, polyhydroxyacetic acid-polyglycol, etc. Their molecular weight is in the range of from 5,000 to 1,000,000 daltons.

Preferably, the pharmaceutically acceptable polymer excipients are selected from PLGA, poly(lactic acid), poly(hydroxy butyrate-hydroxy valerate), etc. Their molecular weight is preferably in the range of from 12,000 to 30,000 daltons.

The above biodegradably pharmaceutically acceptable polymer excipients are more preferably selected from PLGA, poly(acid anhydride), most preferably PLGA.

If PLGA is used, wherein the polymerization ratio of lactide and glycolide is in the range of from 95:5 to 5:95, preferably from 40:60 to 75:25, most preferably about 50:50.

From the view point of maintaining effectiveness for a period of time, biodegradability, and not affecting blood circulation when injected into the body, the sustained-release microspheres of Huperzine A of the invention should have a particle size of from 1 to 250 microns. Too small particle size cannot maintain pharmacological effect for a long time, at the same time, may block blood capillary and affect microcirculation. Too large particle size causes too slow release at initial stage and cannot achieve therapeutically effective blood drug concentration.

In the sustained-release microspheres of the invention, so long as the purpose of sustained-release can be fulfilled, the content of Huperzine A compounds is not especially limited. However, from the viewpoint of ensuring the sustained-release effect of effectively pharmaceutical concentration of blood for sufficient long time and the balance of sustained-release effect, the Huperzine A compounds are preferably present in an amount of from 0.2 to 50% by weight of the microspheres, more preferably not less than 4% by weight; pharmaceutically acceptable polymer excipients are present in an amount of from 50 to 99.8% by weight, preferably not greater than 96% by weight. If the Huperzine A compounds are present in an amount of less than 0.2% by weight, sufficiently high pharmaceutical concentration of blood cannot be obtained. On the contrary, if the amount of Huperzine A compounds is greater than 50% by weight, the stable release of pharmaceuticals may not be assured, which may cause the occurrence of side effects.

The microspheres of the invention can be prepared by conventional methods for making microspheres, such as spray drying method and solvent evaporation method.

When solvent evaporation method is used to prepare the microspheres of the invention, first, Huperzine A compounds and biodegradably pharmaceutically acceptable excipients are dissolved with an organic solvent to obtain an organic phase, in addition, an continuous aqueous phase is prepared with water-soluble pharmaceutically acceptable polymer compounds, then the organic phase is slowly injected through a tubule into the continuous phase, microspheres are obtained under vigorous stiration such as mechanical stiration or ultrasonic stiration, etc. Then the organic solvent is evaporated, the so-obtained microspheres are filtered and dried. If necessary, the microspheres can be washed with water by conventional methods and classified, dried under reduced pressure or lyophilized, and then packaged.

In the above-mentioned operations, Huperzine A compounds and biodegradably pharmaceutically acceptable excipients are as defined above. From the viewpoint of operation, organic solvents should be such organic solvents having sufficient volatility, low residue and low boiling points, for example, dichloromethane, chloroform, ethyl acetate, ether, and mixed solvents of them, etc. The pharmaceutically acceptable polymer compounds for preparing the continuous aqueous phase may be polyvinyl alcohol, carboxymethylcellulose sodium, polyvinylpyrrolidone, sodium poly-methacrylate, sodium polyacrylate, and so on, but are not limited to these.

When preparing the organic phase, the contents of Huperzine A compounds and biodegradably pharmaceutically acceptable excipients in organic solvents are not limited so long as they can be dissolved by organic solvents, but from the viewpoint of the balance between feasible concentration and viscosity and using less organic solvents, the concentrations are preferably from 1 to 30% (w/v). When polyvinyl alcohol, carboxymethylcellulose sodium, polyvinylpyrrolidone, sodium poly-methacrylate, and sodium polyacrylate are used to prepare continuous aqueous phase, their concentrations are not particularly restricted, but based on their solubility in water, their contents in aqueous phase are preferably from 0.01 to 12.0% (w/v), more preferably from 0.01 to 10.0% (w/v), most preferably from 0.1 to 5% (w/v). When the organic phase is injected into the aqueous phase and violently stirred to form microspheres, the volume ratio of the organic phase to the aqueous phase shall be large enough to disperse the organic phase in the aqueous phase completely to form micorpheres having sufficiently tiny particle size and degree of uniformity. But if the aqueous phase is too much, post-treatment is complicated and production cost is increased. From these points, the volume ratio of organic phase to aqueous phase is generally 1:4-100.

The microspheres can also be prepared by spray drying method. When spray drying method is used to prepare sustained-release microspheres of Huperzine A or derivatives thereof, Huperzine A compounds and biodegradably pharmaceutically acceptable excipients are dissolved completely with an organic solvent to form an organic solution, the solution is filtered and prepared into microspheres by conventional spray drying method. If necessary, the microspheres can be performed post-treatment by conventional methods, i.e., washing with water, classifying and packaging.

While the microspheres are prepared by the spray drying method, the organic solvents may be dichloromethane, chloroform, ethyl acetate, dioxane, ether, acetone, tetrahydrofuran, glacial acetic acid, and mixed solvents of them, etc. but are not limited to these.

When preparing the organic phase, the content of pharmaceutically acceptable excipients in organic solvents is not limited so long as the organic solvent can dissolve the excipients, but from the point of the balance of feasible concentration and saving organic solvents, the concentration is preferably from 1 to 30% (w/v).

By comparison of solvent evaporation method and spray drying method for preparing microspheres, from the view point of the degree of uniformity of particle size of the prepared microspheres and easy operation, spray drying method is preferred. From the viewpoint of reducing initial release, solvent evaporation method is preferred.

After the microspheres of Huperzine A or its derivatives of the invention are prepared, the microspheres are classified by particle size or if the particle size is sufficient uniform, the classification may be absent, washed, dried and packaged according to specified dosage, and can be prepared into powder-injection injection and form injection solution in situ upon using. The powder-injection can be directly prepared by the microspheres, and suspended uniformly with physiological saline for injection before using to prepare into injection solution. The microspheres can also be mixed with provided amount of salt, glucose, etc. for isotonic, and to which is added specified amount of pure water for injection to prepare into injection solution before injection. Or alternatively, the microspheres can be suspended based on injection amount and lyophilized, and mixed with water before using.

The method for the treatment of disorders involving acetylcholinesterase and the method for the treatment of Alzheimer's disease according to the invention comprises the step of administration to patients in need of such treatment the injection solution of Huperzine A compounds of the invention. Administration mode is not restricted provided that injection can be used. For examples, intramuscular (im.), subcutaneous (sc.), intradermal (id.), and ventromedial injection, etc can be adopted. From the viewpoint of convenient administration, im. injection is preferred.

Regarding the administration dosage of the sustained-release microspheres of Huperzine A compounds of the invention, taken Huperzine A as an example, for a patient having body weight of 60 kg, calculated by Huperzine A, injection amount once is 1-50 mg, injecting once at least per 15 days. It can be adjusted based on the actual conditions of patients e.g., ages, body weights and disease conditions, etc.

The sustained-release microspheres of Huperzine A compounds according to the invention can achieve sustained-release of Huperzine A compounds. Although it is not limited by any known theories, it could be believed that the mechanism of the sustained-release microspheres according to the invention is that when they are injected into the body, they disperse gradually with blood circulation, in the course of internal circulation, because biodegradable resins such as PLGA do not dissolve in water, but can be gradually degraded by organism, with their gradual degradation, the pharmaceuticals contained in the microspheres are released progressively, thus achieve the sustained-release and long term effect.

The sustained-release microspheres of Huperzine A compounds of the invention can fulfill the durative action of Huperzine A compounds which was not obtained before, and can be administered with, for examples, not less than 10 day, preferably not less than 15 day, more preferably not less than 20 day, even more than 2 month intervals. Therefore, the life quality of patients suffering from Alzheimer's disease can be expected to be greatly improved, and manpower and material resources consumed by quantitative administering on time per day can be reduced, and is very benificial.

### EXAMPLES

The following Examples are provided to illustrate the invention and do not limit it in any way.

The particle size of the microspheres prepared in the following examples were measured by using L2000 type fully-automatic laser granulometer (available from Beckman coulter company). Concentration was determined by high performance liquid chromatograph (HPLC) according to methods disclosed in documents, for examples, Modern Applied Pharmacy Journal (Xiandai Yingyong Yaoxue Zazhi) 1993, 10(1), 51-52; and Zhongguo Yiyao Gongye Zazhi, 1999, 30(8), 363-365.

### Example 1

100 mg Huperzine A and 900 mg PLGA (lactide: glycolide=50:50, molecular weight 13,000) were dissolved in 10 ml dichloromethane, the obtained solution was dripped into 500 ml 0.5% PVA aqueous solution under vigorous stirration (1200 rpm), upon the completion of dripping, the mixture was continuously stirred vigorously for another 3-10 minutes, then the stirring rate was reduced to 300 rpm, the solvent was evaporated for 4-6 hours, filtered, the resulting microspheres were washed with distilled water for three times, and lyophilized. The particle size of the microspheres is 1-200 microns.

### Example 2

To 0.1 g Huperzine A and 2.0 g PLGA (lactide: glycolide=50:50, molecular weight 13,000) was added 30 ml dichloromethane, stirred to completely dissolve, and filtered by using microporous membrane, microspheres were prepared by conventional spray drying method, the particle size was measured to be 1-80 microns, sterilized, and packaged.

The obtained sustained-release microspheres were subjected to releasing test of rabbit in vivo. The dosage was 300 μg/kg, the microspheres were suspended in physiological saline for injection, and injected intramuscularly. From day 1 to day 20, blood sample was taken and measured by HPLC-MS, the blood drug concentration of blood was 0.1-25 ng/ml. The results showed that the sustained-release microspheres of the invention can achieve stable release at least within 20 days.

### Example 3

To 0.2 g Huperzine A and 4.0 g PLGA (lactide: glycolide=50:50, molecular weight 25,000) was added 40 ml dichloromethane, stirred to completely dissolve. The obtained solution was dripped into 500 ml 0.5% PVA aqueous solution under vigorous stirration (1200 rpm), upon the completion of dripping, the mixture was continuously stirred vigorously for another 3-10 minutes, then the stirring rate was reduced to 300 rpm, the solvent was evaporated for 1.5 hours, filtered, the resulted microspheres were washed with distilled water for three times, and lyophilized. The particle size of the obtained microspheres is 1-200 microns.

The so-obtained sustained-release microspheres were subjected to releasing test of dog in vivo. The dosage was 200 μg/kg, the microspheres were suspended in physiological saline for injection, and injected intramuscularly. From day 1 to day 28, blood sample was taken and measured by HPLC-MS, the blood drug concentration was 0.1-5 ng/ml. The results showed that the sustained-release microspheres of the invention can achieve stable release for at least 4 weeks.

### Example 4

To 100 mg Huperzine A and 1.0 g PLGA (lactide: glycolide=75:25, molecular weight 13,000) was added 20 ml glacial acetic acid, stirred to completely dissolve, microspheres were prepared by conventional spray drying method, the particle size was measured to be 1-100 microns, sterilized, and packaged. In vitro analog releasing test was performed for 20 days.

### Example 5

To 0.5 g Huperzine A and 5 g PLGA (lactide:glycolide=50:50, molecular weight 13,000) was added 25 ml dichloromethane, stirred to completely dissolve, the dichloromethane solution was dripped into 500 ml 0.2% carboxylmethylcellulose sodium aqueous solution under vigorous stirration, after the completion of dripping, the mixture was continuously stirred vigorously for another 1 hour and then stirred at 250 rpm for 4 hours, filtered, and lyophilized at low pressure to give microspheres with particle size of 1-150 microns.

### TEST EXAMPLES

In Vitro Release Test of Huperzine A Microspheres

Microspheres as prepared in examples 1 and 4 were used. Releasing test was carried out by simulating in vivo degradation conditions.

According to the inventors' studies, a buffer (citric acid buffer) of certain pH value (pH 4.0 and 5.5) was used, the behavior of pharmaceutical release was similar to that in vivo, therefore, although the pH value was different from the environment in human body, it is generally believed that it can represent the release module in vivo.

Instruments: thermostatic oscillator, centrifuge.

Test conditions: temperature: 37±0.5° C., rate:30 rmp.

Test method: about 10 mg test sample was exactly weighted, placed into plastic centrifugation tube with a lid having a volume of 15 ml, added with 5 ml release medium (citric acid buffer of pH=4 and 5.5) and then placed into a thermostatic oscillator with a certain temperature and rotary rate, sampling on time.

Sampling method: the centrifugation tube was centrifuged at 3600 rpm for 20 min, 3 ml of the solution was exactly sampled, and simultaneously added 3 ml release medium to the centrifugation tube, the liquid was sampled and measured by HPLC method.

Sampling time points (days): 0, 1, 2, 4, 6, 8, 10, 12, 14, 16, 18 and 20, wherein the 0th day means the concentration of pharmaceutical prior to administration at the day of administration.

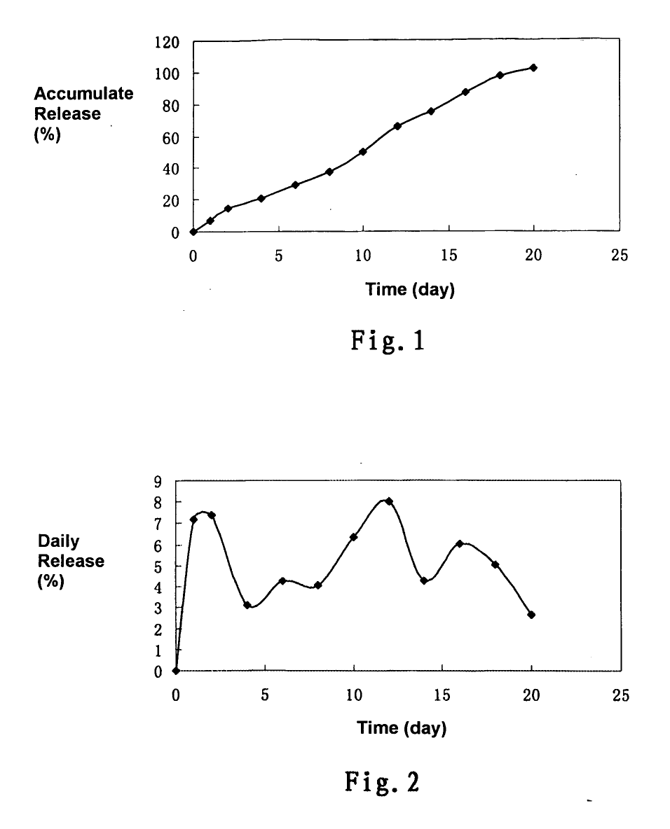

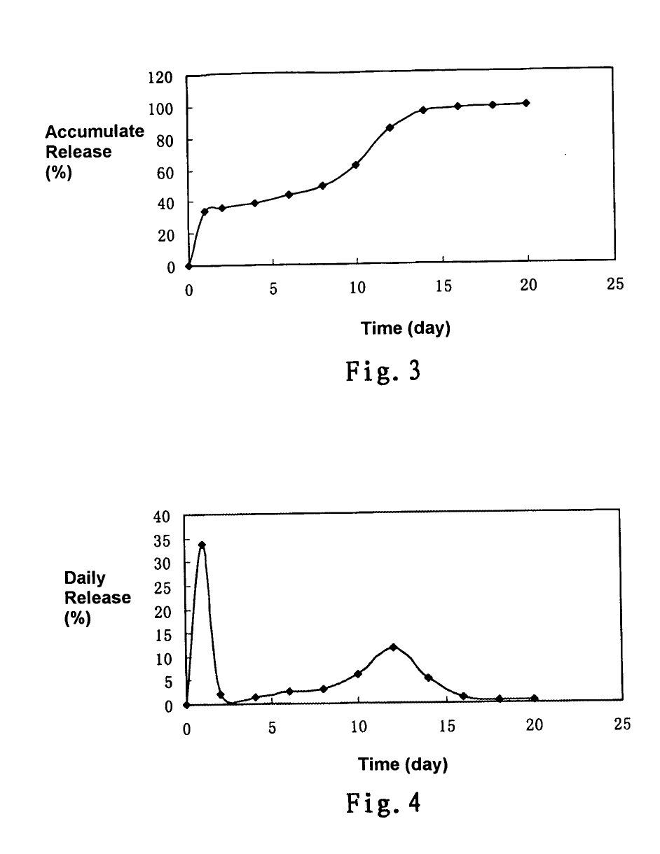

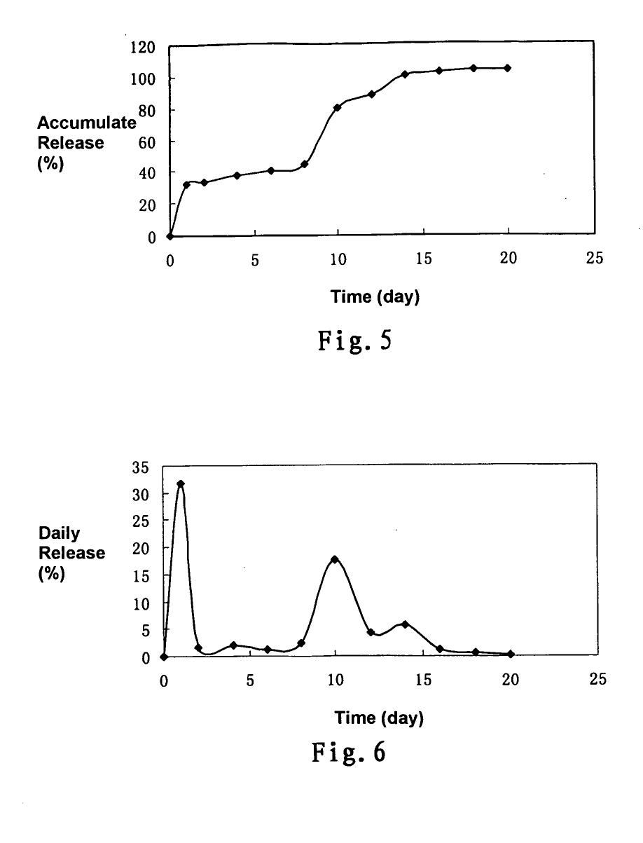

The in vitro release effects of the microspheres prepared in examples 1 and 4 under different pH were shown in FIGS. 1-6, respectively.

The experimental results of the microspheres obtained in examples 1 and 4 were listed in Table 1.

Table 1

Experimental results of in vitro release of sustained-release microspheres of Huperzine A

TABLE 1

| Sample | Drug content | | | Release percentage % | | | | | |
|---|---|---|---|---|---|---|---|---|---|
| No. | μg/mg | pH | Valuing mode | 0 | 1 | 2 | 4 | 6 | 8 |
| Example 1 | 86.5 | 4.0 | The day of sampling | 0 | 7.2 | 7.4 | 3.1 | 4.25 | 4.05 |
| | | | Accumulation | 0 | 7.2 | 14.6 | 20.8 | 29.3 | 37.4 |
| Example 4 | 23.96 | 4.0 | The day of sampling | 0 | 33.8 | 2.1 | 1.3 | 2.5 | 3.0 |
| | | | Accumulation | 0 | 33.8 | 35.9 | 38.5 | 43.5 | 49.5 |
| | | 5.5 | The day of sampling | 0 | 31.7 | 1.7 | 1.95 | 1.3 | 2.35 |
| | | | Accumulation | 0 | 31.7 | 33.4 | 37.3 | 39.9 | 44.6 |

| Sample | Drug content | | | Release percentage % | | | | | |
|---|---|---|---|---|---|---|---|---|---|
| No. | μg/mg | pH | Valuing mode | 10 | 12 | 14 | 16 | 18 | 20 |
| Example 1 | 86.5 | 4.0 | the day of sampling | 6.35 | 8.0 | 4.25 | 6.05 | 5.05 | 2.65 |
| | | | accumulation | 50.1 | 66.1 | 74.6 | 86.7 | 96.8 | 102.1 |
| Example 4 | 23.96 | 4.0 | the day of sampling | 5.95 | 11.65 | 5.05 | 1.2 | 0.45 | 0.35 |
| | | | accumulation | 61.4 | 84.7 | 94.8 | 97.2 | 98.1 | 98.8 |
| | | 5.5 | the day of sampling | 17.65 | 4.2 | 5.75 | 1.15 | 0.55 | 0.2 |
| | | | accumulation | 79.9 | 88.3 | 99.8 | 102.1 | 103.2 | 103.6 |

The release amount of the sampling day in the table is calculated by the accumulated release amount up to the sampling day. In particular, it is estimated that the release rate of drugs at the interval of two tests is not change. By represented with a formula, intraday release amount=(accumulated release amount up to the sampling day−accumulated release amount measured by the last time)/interval days between the present test and last test.

For example, in Example 1, the release amount at the 0th day is 0, the accumulated release amount at the first day is 7.2, the intraday release amount at the first day=(7.2−0)/(1−0)=7.2. The release amount at the second day is 14.6, the accumulated release amount at the second day=(14.6−7.2)/(2−1)=7.4. The release amount at the fourth day is 20.8, and the accumulated release amount at the fourth day=(20.8−14.6)/(4−2)=3.1. The rest may be deduced by analogy.

It can be understood that the above accumulated release amount over 100% is caused by the accumulating magnification of test errors.

It can be seen from the above table, the release of sustained-release microspheres of Huperzine A of the invention is stable in longer than 20 days. Therefore, for patients suffering from Alzheimer's disease, administration frequency can be greatly reduced, at the same time, the dosage is effectively controlled, and the occurrence of side effect is avoided.

### INDUSTRIAL APPLICAPABILITY

The invention uses biodegradably polymer to embed Huperzine A compounds so as to prepare microsphere formulation for injection, once injection can maintain effectious over a period of 15 days or longer. For patients suffering from Alzheimer's disease and other disorders involving acetylcholinesterase, undoubtedly, it is excellent evangel.

1. A sustained-release microsphere of Huperzine A compounds, characterized in that the sustained-release microsphere is consisted of a Huperzine A compound represented by the formula (Ia) and a biodegradably pharmaceutically acceptable polymer excipient, 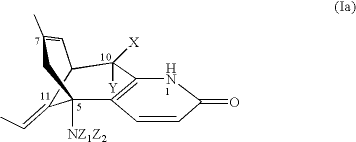
wherein, X and Y independently represent hydrogen or methyl, Z₁ and Z₂
independently represent hydrogen or in combination represent

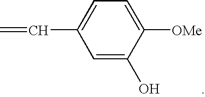

2. The sustained-release microsphere according to claim 1, wherein the Huperzine A compound is compound (I), (II), (III), (IV) or (V) or a salt thereof consisting of an organic acid or inorganic acid selected from hydrochloric acid, acetic acid, phosphoric acid, sulfuric acid, lactic acid and citric acid, wherein the compound (I), (II), (III), (IV) and (V) are Huperzine A, N5-(3′-hydroxy-4′-methoxy-phenylmethylene) Huperzine A, (10S)-10-methyl Huperzine A, (10R)-10-methyl Huperzine A, and 10,10-dimethyl Huperzine A, respectively.

3. The sustained-release microsphere according to claim 1 or 2, wherein the pharmaceutically acceptable polymer excipient is selected from poly(D, L-lactide-co-glycolide) (PLGA), poly(lactic acid), poly(glycollic acid), poly(3-hydroxy butyrate), polylactone, poly(acid anhydride), poly (hydroxy butyrate-hydroxy valerate) copolymer, poly(acrylic glucosan), poly (lactic acid)-polyethyleneglycol, poly(hydroxyacetic acid)-poly ethylene glycol or a mixture thereof.

4. The sustained-release microsphere according to claim 3, wherein the pharmaceutically acceptable polymer excipient is selected from PLGA, poly(lactic acid), poly(hydroxy butyrate-hydroxy valerate) copolymer or a mixture thereof.

5. The sustained-release microsphere according to claim 4, wherein the pharmaceutically acceptable polymer excipient is PLGA.

6. The sustained-release microsphere according to claim 5, wherein said PLGA has a molecular weight of from 12,000 to 30,000 daltons.

7. The sustained-release microsphere according to claim 5 or 6, wherein the polymerization ratio of lactide to glycolide in PLGA is in the range of from 95:5 to 5:95.

8. A method for preparing sustained-release microsphere of Huperzine A compound, comprising dissolving the Huperzine A compound according to claim 1 or 2 and the biodegradably pharmaceutically acceptable excipient according to any of claims 3-7 with an organic solvent, dropping the organic solvent phase into a continuous aqueous phase prepared with a pharmaceutically acceptable polymer to form microspheres, evaporating the organic solvent, filtering to give sustained-release microspheres.

9. The method according to claim 8, characterized in that the organic solvent is selected from dichloromethane, chloroform, ethyl acetate, ether, or a mixture thereof, the biodegradably pharmaceutically acceptable excipient is present in the organic solvent in an amount of from 1 to 30% (w/v), and/or the pharmaceutically acceptable water-soluble polymer is selected from polyvinyl alcohol, carboxymethylcellulose sodium, polyvinylpyrrolidone, sodium polymethacrylate, sodium polyacrylate, the content in the aqueous phase of which is 0.1-5 (w/v).

10. A method for preparing sustained-release microsphere of Huperzine A compound, comprising dissolving a Huperzine A compound and biodegradably polymer excipient with an organic solvent, and microspheres are prepared by spray drying method.

11. The method according to claim 10, wherein the organic solvent is selected from dichloromethane, trichloromethane, ethyl acetate, dioxane, ethyl ether, acetone, tetrahydrofuran, glacial acetic acid.

12. A method for the treatment of Huperzine A or disorders involving acetylcholinesterase, comprising administering therapeutically effective amount of the sustained-release microspheres according to any of claims 1-7 or the sustained-release microspheres prepared by the method according to any of claims 8-11 by injection to patients in need of such treatment at a determined period of not less than 10 days interval.
